(19) United States
(12) Patent Application Publication
(10) Pub. No.: US 2017/0065952 A1
(43) Pub. Date: Mar. 9, 2017

(54) PICKERING EMULSION FORMULATIONS

(71) Applicant: THE UNIVERSITY OF BIRMINGHAM, West Midlands (GB)

(72) Inventors: Fotios SPYROPOULOS, West Midlands (GB); Aleksandra PAWLIK, West Midlands (GB); Daniel KURUKJI, West Midlands (GB); Ian NORTON, West Midlands (GB)

(21) Appl. No.: 15/308,909

(22) PCT Filed: May 6, 2015
(86) PCT No.: PCT/GB2015/051333

§ 371 Date: Nov. 4, 2016

(30) Foreign Application Priority Data
May 6, 2014 (GB) 1407934.7

(51) Int. Cl. B01F 17/00; A61K 8/06; A61K 9/107; A23L 29/10; A61K 8/73; A61K 47/42; A61K 8/64; A01N 25/04; A61K 47/36

(52) U.S. Cl. CPC B01F 17/0085; A01N 25/04; A61K 8/062; A61K 8/064; A61K 9/107; A61K 47/36; A61K 8/736; A61K 47/42; A61K 8/64; B01F 17/0021; B01F 17/005; A23L 29/10; A61K 2800/10; A23V 2002/00

(57) ABSTRACT

The invention provides an emulsion comprising: (a) a continuous phase; (b) a dispersed phase comprising an active compound (1); and (c) colloidal particles located at the interface between the continuous phase and the dispersed phase. These are particularly useful with one or more agrochemicals or food additives as an active compound.

The present invention relates to solid-stabilised emulsions, to processes for preparing said emulsions and to methods of using said emulsions. The emulsions comprise a continuous phase; a dispersed phase comprising an active compound; and colloidal solid lipid particles located at the interface between the continuous phase and the dispersed phase. The continuous phase can be aqueous when the dispersed phase is an oil phase or the continuous phase can be an oil phase when the dispersed phase is an aqueous phase. The colloidal solid lipid particles are pre-formed particles.

### BACKGROUND OF THE INVENTION

In order to achieve stable dispersions of one liquid in another, emulsions in the traditional sense require the addition of an interface-active substance (emulsifier). Emulsifiers have an amphiphilic molecular structure, consisting of a polar (hydrophilic) and a nonpolar (lipophilic) molecular moiety, which are spatially separate from one another. In simple emulsions, finely disperse droplets of one phase, surrounded by an emulsifier shell (water droplets in water-in-oil [W/O] emulsions or lipid vesicles in oil-in-water [O/W] emulsions) are present in the second phase. Emulsifiers lower the interfacial tension between the phases by positioning themselves at the interface between the two liquids. At the phase boundary, they form oil/water interfacial films, which prevent irreversible coalescence of the droplets. Emulsions are frequently stabilized using emulsifier mixtures.

Oil based formulations (oil as the continuous phase) are obtained by dissolving, emulsifying and/or suspending active materials in an oil phase. Such products are employed across a range of technology and business sectors that includes pharmaceutical agents, food additives, cleaning agents, complexing agents, personal care substances, lubricants, adhesives, heating/cooling agents, colourants, indicators and crop protection chemicals. Water-in-oil emulsions (oil continuous emulsions) contain water soluble active materials in the dispersed aqueous phase. Water-based formulations (water as the continuous phase) are obtained by dissolving, emulsifying and/or suspending active materials in water. The use of such products is widespread across many technology and business sectors but includes pharmaceutical agents, food additives, cleaning agents, complexing agents, personal care substances, lubricants, adhesives, heating/cooling agents, colourants, indicators and crop protection chemicals. Within crop protection, the efficient use of aqueous systems with certain crop protection agents, however, may be restricted due to their poor water-solubility. Suspensions are only appropriate for high melting point solid a.i.s. Therefore active agents are often administered in the form of oil-in-water emulsions. These aqueous continuous phase systems (oil-in water emulsions) containing liquid, substantially water-insoluble pesticide technical materials may be formulated as emulsions or suspoemulsion formulations comprising low molecular weight or polymeric surfactants either alone or in admixture. However, these formulation types can suffer from a variety of problems including droplet coalescence followed by phase separation under the influence of temperature variations or due to the presence of high electrolyte concentrations either in the formulation or in the medium used to dilute the formulation prior to spray application. The presence of an emulsified oil phase increases the risk of formulation failure due to the intrinsic instability of oil-in-water emulsions. Due to the relatively complex supply chain for crop protection agents, formulated products may be stored for long periods and may be subjected during storage and shipping to extreme temperature variations, high-shear and repetitive vibration patterns which can increase the likelihood of failure.

It may often be desirable to combine different agrochemicals to provide a single formulation (taking advantage of the additive properties of each separate agrochemical or food additive) and optionally an adjuvant or combination of adjuvants that provide optimum biological performance. In commercial practice it is often desired to minimize transportation and storage costs by using a formulation in which the concentration of the active agrochemical(s) in the formulation is as high as is practicable and in which any desired adjuvants are “built-in” to the formulation as opposed to being separately tank-mixed. The higher the concentration of the active agrochemical(s) however, the greater is the probability that the stability of the formulation may be disturbed and one or more components separate out.

In general, the separation of a component from an agrochemical formulation is highly undesirable, particularly when the formulation is sold in bulk containers. In these circumstances it is difficult to re-homogenize the formulation and to achieve even distribution of the components on dilution and spraying. Furthermore, the formulation must be stable in respect of storage for prolonged periods in both hot and cold climates. These factors present formidable problems to the formulator. The problems may be exacerbated still further if the formulation contains a water-soluble agrochemical electrolyte and a second agrochemical system which is substantially water-insoluble.

In the early 1900s, Pickering prepared paraffin/water emulsions that were stabilized merely by the addition of various colloidal solids, such as basic copper sulphate, basic iron sulphate or other metal sulphates. This type of emulsion is thus also referred to as a Pickering emulsion. For this type of emulsion, Pickering postulated the following conditions:

(1) The solid particles are only suitable for stabilization if they are significantly smaller than the droplets of the inner phase and do not have a tendency to form agglomerates.
(2) An important property of an emulsion-stabilizing colloidal solid is also its wettability. For example, in order to stabilize an O/W emulsion, the colloidal solid has to be wettable by water and by oil. Particles may be located at the interface as a monolayer or multilayer.

The original forms of Pickering emulsions initially surfaced as undesired secondary effects in a variety of industrial processes, such as, for example, in secondary oil recovery, the extraction of bitumen from tar sand and other separation processes involving two immiscible liquids and fine, dispersed solid particles. These are generally W/O emulsions which are stabilized by mineral solids. Accordingly, investigation of corresponding systems, such as, for example, the oil/water/soot or oil/water/slate dust systems was initially the focus of research activity.

Basic experiments have shown that one characteristic of a Pickering emulsion is that the solid particles are arranged at the interface between the two liquid phases where they form, as it were, a mechanical barrier against the coalescence of the liquid droplets.

Pickering emulsions are encountered in various natural and industrial processes such as crude oil recovery, oil separation, cosmetic preparation, and waste water treatment.

Advantages of formulating compositions such as pesticidal compositions and food additives as a Pickering emulsion include:
1. Stability of the emulsion to coalescence over a broad range of storage temperatures, e.g. from the freezing point up to at least 50° C. and normally 80° C. or higher.
2. Simplicity in that instead of a mixture of 2 or more emulsifiers selected from the 1000 or more commercial surfactants available, Pickering emulsions are formed from normally only 1 but occasionally 2 colloidal solids selected from the limited number of commercially practical options.
3. Stability of the emulsion to coalescence over a broad range of pH and electrolyte conditions, including where the high electrolyte concentration may be due to one of the active ingredients, e.g. Pickering emulsions typically do not coalesce or exhibit any oil separation when diluted into simple nitrogen or complex (e.g., sulphur mixture) fertilizers.
4. Pickering emulsions may slowly form sediment upon dilution, as do many other formulations, but they re-suspend extremely easily and are therefore more convenient when left for example in a storage tank overnight without agitation.

### SUMMARY OF THE INVENTION

We have now found that solid lipid particles, or coaservates, in the form of “solid lipid-dispersions” can be prepared and used as colloidal particles to prepare and stabilize an emulsion where such colloidal particles can contain an active agent dissolved or embedded within the solid lipid particles.

There is now provided emulsion compositions and to methods of using said emulsions. In one aspect, the present invention relates to an emulsion comprising
(a) a continuous phase;
(b) a dispersed phase comprising an active compound (1); and
(c) colloidal particles located at the interface between the continuous phase and the dispersed phase.

Pickering stabilisation is believed to occur as a result of the association or adsorption of an assembly (e.g. the lipid crystals or a dynamic structure such as complexes) into an emulsion interface. The result of this association/adsorption is the formulation of a protecting layer around droplets (substantially) preventing coalescence and increasing/promoting ability.

The assembled structure is believed to be colloidal in nature.

A number of ways of triggering release are shown in examples below including pH and temperature. Other releases include osmotic stress, mechanical stress, ionic conditions and radiation such as light.

The colloidal particles may be solid lipid particles. These particles, such as solid lipid particles, may be present at the emulsion interface or elsewhere in the system as discrete particles. Typically they are present at the emulsion interface.

Advantages include:
(a) controlling flavour release (e.g. taste making)
(b) protecting the active against chemical/photochemical or physical degradation during manufacture, storage and/or end-use
(c) to control/trigger/modify/target delivery of the active at a defined site
(d) reduce volatility of active components.

The continuous phase may be aqueous when the dispersed phase is an oil phase or the continuous phase can be an oil phase when the dispersed phase is an aqueous phase. The colloidal solid lipid particles are pre-formed. In the case of an oil-in-water emulsion, it comprises a colloidal solid lipid particle and a dispersed phase comprising at least one active ingredient which is either itself an oily liquid comprising the oil phase, is a solid but is dissolved in an oily liquid present in the oil phase, is a solid and is dispersed within the oil phase or is present as a colloidal solid adsorbed to the liquid-liquid interface between the continuous aqueous phase and the dispersed oil phase. In the case of a water-in-oil emulsion, it comprises a colloidal solid lipid particle and a dispersed aqueous phase comprising at least one active ingredient which is dissolved in water is a solid and is dispersed within the aqueous phase or is present as a colloidal solid adsorbed to the liquid-liquid interface between the continuous oil phase and the dispersed aqueous phase.

### DETAILED DESCRIPTION OF THE INVENTION

The emulsion of the present invention comprises
(a) a continuous phase;
(b) a dispersed phase comprising an active compound (1); and
(c) colloidal particles located at the interface between the continuous phase and the dispersed phase.

The continuous phase can be aqueous when the dispersed phase is an oil phase or the continuous phase can be an oil phase when the dispersed phase is an aqueous phase. The colloidal particles are pre-formed.

The colloidal particles may be fully or partially fused particles.

The particles may be fully or partially associated particles.

Active compound (1) is itself the dispersed phase, is dissolved in the dispersed phase, is suspended in the dispersed phase or is present as a colloidal solid at the interface of the dispersed phase and the continuous phase.

In the case of an oil-in-water emulsion, it is a colloidal solid lipid particle stabilized oil-in-water emulsion comprising colloidal solid lipid particles and a dispersed emulsion phase comprising at least one active compound which is either itself an oily liquid comprising the oil phase, is a solid but is dissolved in an oily liquid present in the oil phase, is a solid and is dispersed within the oil phase or is present as a colloidal solid adsorbed to the liquid-liquid interface between the continuous aqueous phase and the dispersed oil phase.

In the case of a water-in-oil emulsion, it is a colloidal solid lipid particle stabilized, water-in-oil emulsion comprising colloidal solid lipid particles and a dispersed emulsion phase comprising at least one active compound which is dissolved in water, is a solid and is dispersed within the aqueous phase or is present as a colloidal solid adsorbed to the liquid-liquid interface between the continuous oil phase and the dispersed aqueous phase.

The solid lipid dispersion may be conveniently prepared by a melt emulsification process The solid lipid dispersion may be conveniently prepared by a melt emulsification process whereby the molten lipid is added to an aqueous phase to form a lipid-in-oil emulsion followed by subsequent cooling of the emulsion to below the melting point of the lipid and solidification of the lipid to produce the solid lipid dispersion. Other methods may also be employed to produce the solid lipid dispersion.

The colloidal solid is present as a solid lipid dispersion that may contain a further active compound that may or may not be the same as the active compound contained within the emulsion droplet.

This allows for dual and independent release of active compounds from the different locations on the emulsion droplet.

By incorporation of active compounds into both the stabilising particles and the internal structure of the emulsion it is possible to achieve triggered or controlled delivery of two segregated active compounds (e.g. hydrophobic active-1 and hydrophilic active-2) from a single structured simple emulsion potentially over different time scales.

Each active compound may be selected from agrochemicals (which include pesticidally active ingredients, plant growth regulators, plant activators, safeners and bioperformance enhancing adjuvants), pharmaceuticals, food additives, cleaning agents, personal care substances, colourants, complexing agents, adhesives, heating/cooling agents or indicators.

The active compound may be a food additive. It may be a pharmaceutical such as an unpleasant tasting compound and be used to mask the taste of the compound.

Incorporation of both hydrophilic and hydrophobic molecules within one structured simple emulsion is possible, triggered/controlled release of active compounds may be independently controlled so the potential exists to release these over the same or indeed different timescales. Release may be better controlled by careful formulation of the particles themselves and/or the emulsion structure they are used to stabilise. Release can be triggered by a range of different “stimuli” including enzymatically, change in temperature, pH or ionic conditions. Different active ingredients may use different release stimuli. The need for added amounts of emulsifier(s) needed to stabilise current systems is greatly reduced or even eliminated. The formulated smart emulsion structures may be manufactured using techniques already in operation at larger scales and thus industrial feasibility is achievable. Especially, the invention provides for the preparation of stable mixtures of active compounds that would be physically of chemically incompatible when mixed directly in a normal formulation and also provides the ability to produce a product with a range of release characteristics (from both the dispersed oil phase and the solid lipid particle stabilising layer).

In a further embodiment, it is possible to encapsulate the emulsion droplet within a shell of solid lipid (containing a further active compound) by raising the temperature of the system to above the melting point of the solid lipid composite and allowing the lipid particles to fuse/melt around the emulsion droplet. Cooling then solidifies the solid lipid producing a robust microcapsule.

In yet a further embodiment, where the continuous phase is aqueous and the dispersed phase is an oil, it is possible to encapsulate the oil dispersed phase by incorporating reactive polymers, pre-polymers or monomers within the oil dispersed phase and after formation of the initial particle stabilised emulsion, allowing the reactive component to react at the interface by a further added component (or one produced from within) to encapsulate fully the contents of the oil droplet, thus improving the ability to control the release of contents of the product. This includes any polymerisable substance that can be polymerized in an emulsion. These substances may include a polyurethane precursor such as a diol, a diisocyanate and/or a monomer containing both alcohol and isocyanate functional groups. They may also include a polyurea precursor such as an isocyanate and/or an amine such as a diamine or a triamine. Examples of isocyanates include diisocyanates (such as toluene diisocyanate, hexamethylene diisocyanate and isophorone diisocyanate); isocyanates with, on average, more than two isocyanate groups (such as polymethylenepolyphenylene isocyanate); and many others including prepolymers of diisocyanates such as their reaction products with trimethylol propane and other simple polyols sold as Desmodur™ resins from Bayer. Alternatively they may also include a polyamide precursor such as an acid chloride and/or a triamine.

In one preferred embodiment, the organic phase contains at least one diisocyanate and/or polyisocyanate, whilst the aqueous phase contains at least one diamine and/or polyamine.

Any diisocyanate or polyisocyanate, or mixtures thereof, may be employed, provided that it is soluble in the liquid chosen for the organic phase. Where aromatic liquids are used, aromatic isocyanates such as isomers of tolylene diisocyanate, isomers and derivatives of phenylene diisocyanate, isomers and derivatives of biphenylene diisocyanates, and/or polymethylenepolyphenyleneisocyanates (PMPPI) are suitable. Where aliphatic liquids are used, aliphatic isocyanates are suitable, for example aliphatic acyclic isocyanates such as hexamethylenediisocyanate (HMDI), cyclic aliphatic isocyanates such as isophoronediisocyanate (IPDI) or 4,4′methylenebis(cyclohexyl isocyanate), and/or trimers of HMDI or IPDI. and the like. Polymeric polyisocyanates, biurets, blocked polyisocyanates, and mixtures of polyisocyanates with melting point modifiers may also be used. MDI is a particularly preferred polyisocyanate. Should other properties be desired from the isocyanate such as increased flexibility, then pegylated derivatives may be employed wherein part of the isocyanate is reacted with a suitable polyol. Such techniques and chemistries are well known in the art.

The concentration of the isocyanate(s), and the ratio(s) where more than one isocyanate is used, is/are chosen so as to obtain the desired release rate profile for the particular end application.

The diamine or polyamine, or mixtures thereof, may be any such compound(s) which is/are soluble in the aqueous phase. Aliphatic or alicyclic primary or secondary diamines or polyamines are very suitable, such as ethylene-1,2-diamine, diethylenetriamine, triethylenetetramine, bis-(3-aminopropyl)-amine, bis-(2-methylaminoethyl)-methylamine, 1,4-diaminocyclohexane, 3-amino-1-methylaminopropane, N-methyl-bis-(3-aminopropyl)amine, 1,4-diamino-n-butane, 1,6-diamino-n-hexane and tetraethylenepentamine. Polyethyleneimines are also suitable.

The molar ratio of amine moieties to isocyanate moieties may be varied from about 0.1:1 to about 1.5:1. Suitably either (i) approximately equimolar concentrations of amine and isocyanate moieties are employed, with the molar ratio of amine to isocyanate moieties ranging from about 0.8:1 to about 1.3:1; or (ii) a significant excess of isocyanate is present, with the molar ratio of amine to isocyanate moieties ranging from about 0.1:1 to about 0.35:1.

Other wall chemistries may be used, for example polyurethanes and polyamides, by appropriate selection of wall forming components. Suitable glycols for addition through the aqueous phase include those taught above and which are water soluble. These may also include simple polyhydroxylic glycols, for example, suitable diols are ethylene glycol, 1,2-butanediol, diethylene glycol, triethylene glycol, polyalkylene glycols, such as polyethylene glycol, and also 1,2- and 1,3-propanediol, 1,4-butanediol, 1,6-hexanediol, neopentyl glycol or neopentyl glycol hydroxypivalate. Examples of polyols having 3 or more hydroxyl groups in the molecule, which may be used additionally, if desired, include trimethylolpropane, trimethylolethane, glycerol, erythritol, pentaerythritol, di-trimethylolpropane, dipentaerythritol, trimethylol-benzene and trishydroxyethyl isocyanurate. Higher functionality may be employed by use of the various sugars such as fructose, dextrose, glucose and derivatives thereof. Mixtures of water soluble and oil soluble reactive hydroxyl containing compounds are also contemplated. Polyamides may be produced in a similar manner by selection of an appropriate acid feedstock (such as sebacoyl chloride). Mixtures, in any ratio, of polyureas, polyurethanes and polyamides are also part of the present invention. Therefore suitably the polymeric shell is a polymer which is a polyurea, a polyamide or a polyurethane or is a mixture of two or more of these polymers; more suitably it is a polyurea.

The particles may comprise a coating or complex of one or more polysaccharides, one or more proteins or a mixture of one of more polysaccharides and one or more proteins. Examples of such materials include lactoglobulin, pectins (such as low methoxyl pectins) and waxes.

This then allows the preparation of a triggerable system wherein the particles such as colloidal solid lipid particles may be induced to act as the trigger for release of an active compound by external stimuli (such as temperature, change in pH and hydrolysis).

The range of active compounds that may be incorporated into either the dispersed phase or the colloidal stabilizing solid lipid particles is only limited by the suitability of an active compound to be formulated into either phase. When present, the ratio of active compounds in the two phases may be varied by the individual loadings in the dispersed phase or in the colloidal solid lipid particle, the size of both the colloidal solid lipid particle and the emulsion droplet and the ratio of stabilising particle to dispersed phase.

Although there is no necessity to add further surfactants as either emulsification aids or dispersion aids, these may be added, when the emulsion is further processed to produce a fully encapsulated product.

For agrochemicals (such as pesticidally active ingredients) employed in oil-in-water emulsions, in the event that more than one water-insoluble agrochemical is present in the compositions of the present invention, it is understood that each water-insoluble agrochemical, independently, is either itself an oily liquid comprising the oil phase, is a solid but is dissolved in an oily liquid present in the oil phase, is a solid and is dispersed within the oil phase or is present as a colloidal solid adsorbed to the liquid-liquid interface between the continuous aqueous phase and the dispersed oil phase.

The pesticidally active ingredient may be any known in the art. The term “pesticidally active” refers to chemicals and biological compositions, such as those described herein, which are effective in killing, preventing or controlling the growth of undesirable pests, such as, plants [generally weeds], insects, mice, microorganisms, algae, fungi, bacteria, and the like. The term “agrochemical” may also apply to compounds that control the growth of plants in a desired fashion (e.g. growth regulator), to a compound which mimics the natural systemic activated resistance response found in plant species (e.g. plant activator) or to a compound that reduces the phytotoxic response to a herbicide (e.g. safener). In this context, the term “agrochemical” also applies to additives to a product that can affect the activity of a pesticidally active ingredient—such as an oil or surfactant. These additives are known collectively as “adjuvants”. The pesticidally active ingredients are independently present in an amount that is biologically effective when the composition is diluted, if necessary, in a suitable volume of liquid carrier, e.g. water, and applied to the intended target, e.g. the foliage of a plant or locus thereof or incorporated into or coated onto materials, such as building materials or used for treating hides, for example, in the leather tanning process.

In a further aspect then, the emulsions of the present invention may be used to kill, prevent or control the growth of a pest.

Dispersed as an emulsion in the continuous aqueous phase is an organic phase containing a substantially water-insoluble agrochemical, sometimes referred to herein for brevity as a “water-insoluble” agrochemical even if it has measurable solubility in water. This agrochemical preferably has a solubility in water at 20° C. not greater than about 5000 mg/l as measured at the pH of the aqueous phase of the agrochemical composition. It will be apparent to one skilled in the art that the solubility in water of some agrochemical depends on pH if they have a titratable acid or base functionality; specifically acids are more soluble above their pKₐ and bases are more soluble below their pK_{b}. Thus acids may be rendered insoluble in water for the purposes of the present discussion if the aqueous phase is maintained at a pH close to or below their pKₐ, even if they may be more soluble than about 5000 mg/l at a higher pH. Especially preferred water-insoluble agrochemicals useful in the present invention have a solubility in the aqueous phase at 20° C. not greater than about 2000 mg/l. In certain circumstances as described below the water-insoluble agrochemical can itself serve as the colloidal solid, in which case the solubility at 20° C. of the agrochemical must be below about 100 mg/l in both the aqueous and disperse phases.

The substantially water-insoluble agrochemical or a mixture of agrochemicals can be liquid at ambient temperature or can be liquified by warming, or can be dissolved in a suitable solvent, or can be dispersed as solids in a suitable water-immiscible liquid, or can be adsorbed to the liquid-liquid interface as a colloidal solid, and is/are substantially insoluble in water.

In an embodiment of the invention, the oil phase comprises a liquid with intermediate hydrophobicity so that it does not substantially dissolve or become miscible with water and is not so hydrophobic that the colloidal solids are unable to efficiently contact both the oil and water phases and thus remain at the interface. Preferably, the oil phase has a log octanol-water partition coefficient (or log P) above 1 and below 7, preferably below 3.

In one embodiment, the oil droplets have a volume-weighted median diameter as measured by laser light scattering of 100 micron or less.

In the event that the substantially water-insoluble agrochemical is a high viscosity liquid or a solid, a solvent may be used to dissolve the substantially water-insoluble agrochemical and form a low viscosity liquid.

The solvent must be substantially immiscible with water and the affinity of the solvent for the agrochemical present in the disperse oil phase must be such that substantially all of the agrochemical is partitioned in the oil phase and substantially none is partitioned in the aqueous phase. One skilled in the art will readily be able to determine whether a particular organic solvent meets this second criterion for the agrochemical in question by following any standard test procedure for determining partition of a compound (in this case, the oil-soluble or miscible or oil-dispersed agrochemical) between water and the organic solvent. For example, one such test procedure comprises the following steps.
1. A solution of the oil-soluble or miscible agrochemical is prepared in the organic solvent at as high a concentration as possible;
2. An aliquot of 10 g of this solution is added to 90 g water in a glass bottle, which is shaken on a mechanical shaker for 4 hours at ambient temperature;
3. The contents of the glass bottle are permitted to phase separate for 4 days;
4. Subsamples of the resulting oil and water phases are taken and analysed by HPLC to determine concentrations C_{O} and C_{W} in the oil and water phases respectively. The subsample of the water phase is preferably centrifuged before analysis to remove traces of organic solvent; and
5. A partition coefficient, analogous to octanol-water partition coefficient P, is calculated as C_{O}/C_{W}. The partition coefficient is conveniently expressed as a logarithm.

In some cases the concentration of the agrochemical in the water phase will be below the detection limit of the HPLC method. In other cases, traces of the organic solvent are found in the water phase, even after centrifugation, so that the apparent concentration of oil-soluble or miscible or oil-dispersed agrochemical observed in the water phase is misleadingly high. In such cases, a published value for solubility in water of the oil-soluble or miscible or oil-dispersed agrochemical in question can be used in place of C_{W} for calculation of the partition coefficient.

The solvent is selected such that the agrochemical exhibits a partition coefficient such that log(C_{O}/C_{W}) is about 2 or greater, preferably about 3 or greater. Preferably the agrochemical is soluble in the organic solvent by at least about 5% by weight, more preferably by at least about 10% by weight and most preferably by at least about 15% by weight. Generally, organic solvents having a higher solubility for the agrochemical therein are more suitable, provided the organic solvent is substantially immiscible with water, i.e., the organic solvent(s) remains as a separate liquid phase from the aqueous phase at 20° C. when mixed at ratios between about 1:100 up to about 100:1.

Organic solvents useful in compositions of the present invention preferably have a flash point above about 35° C., more preferably above about 90° C., and are preferably not antagonistic to the biological effectiveness of any of the agrochemicals of the composition. Moreover, these solvents must not significantly affect the physical form of the solid lipid colloidal stabilising particle. Examples of suitable solvents for use in the present invention include petroleum derived solvents such as mineral oils, aromatic solvents and paraffins. Naphthalenic aromatic solvents such as Solvesso™ 100, Solvesso™ 150 or Solvesso™ 200, commercially available from Exxon Mobil Chemical of Houston, Tex. and alkyl acetates with high solvency, such as Exxate™ 1000, also available from Exxon Mobil Chemical. Useful aromatic solvents include benzene, toluene, o-xylene, m-xylene, p-xylene, mesitylene, naphthalene, bis-(α-methylbenzyl)xylene, phenylxylene and combinations thereof. Other useful solvents include substituted aromatic solvents such as chlorobenzene or ortho-dichlorobenzene. Further solvents suitable for preparing the oil phase include alkyl ketones, methyl esters of fatty acids derived from fats and oils such as methyl oleate, n-octanol, alkyl phosphates or phosphonates such as tri-n-butyl phosphate, tri-2-ethylhexyl phosphate or bis-2-ethylhexl-2-ethyl hexyl phosphonate, fatty acid alkyl amides such as Agnique™ KE3658 available from Cognis of Cincinnati, Ohio or Hallcomid™ M-8-10 available from Stepan Chemical of Northfield Ill.

The water-insoluble agrochemicals may, themselves, comprise the oil phase, may be solubilized in a hydrophobic solvent to form the oil phase, may form the colloidal solid, and/or may be dispersed within the oil phase. Depending upon the solvent selected, an agrochemical may be solubilized or dispersed in the oil phase, or adsorbed to the interface between the oil and aqueous phases of the present invention.

The substantially water-insoluble agrochemicals having solubility in the aqueous phase at 20° C. of not greater than about 5000 mg/l, more preferably not greater than about 2000 mg/l, and including plant growth regulators, herbicides, (herbicide) safeners, insecticides and fungicides, suitable for use in the present invention include:
A. Agrochemicals that below about 20° C. are liquids or that remain stable for at least several days as liquids and which themselves comprise the oil phase alone, or are used in combination with an organic solvent substantially immiscible with the aqueous phase. Examples of pesticidally active ingredients of this type include metolachlor, S-metolachlor, permethrin and propiconazole.
B. Agrochemicals that have melting points between about 20° C. and about 80° C. that can be melted and then formed into an emulsion. Examples of pesticidally active ingredients of this type include cyprodinil, lambda cyhalothrin and myclobutanil.
C. Solid agrochemicals (at ambient temperature) that are soluble at 20° C. to a concentration of at least about 50,000 mg/l and more preferably at least about 150,000 mg/l in an organic solvent substantially immiscible with the aqueous phase. Examples of pesticidally active ingredients of this type include abamectin, clodinafop and lambda cyhalothrin.
D. Solid agrochemicals (at ambient temperature) that may be dispersed and retained within the oil phase include any agrochemicals having a melting point above about 50° C. and that have solubility at 20° C. of below about 5000 mg/l, more preferably below about 2000 mg/l, in the oil phase. Representative solid pesticidally active ingredients include chlorothalonil, isoxaflutole, mesotrione, including salts and chelates thereof, PPO inhibitors such as butafenacil, prodiamine, triazines such as atrazine, simazine and terbuthylazine, sulfonylurea herbicides such as primisulfuron, prosulfuron, azoxystrobin, fludioxonil, thiabendazole and a compound of the formula (I), described in U.S. Pat. No. 6,537,948:

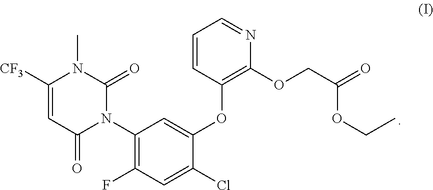 [SMILES: CCOC(=O)COc1ncccc1Oc1cc(-n2c(=O)cc(C(F)(F)F)n(C)c2=O)c(F)cc1Cl]

For the purposes of this embodiment, solid agrochemicals include those that substantially remain in solid form dispersed in the oil phase. The solid agrochemicals may exhibit limited solubility in a solvent present in the oil phase but not commercially useful levels of solubility in commercially useful solvents or which may be readily soluble in certain solvents, but which solvents either are not present in the oil phase or not present in an amount sufficient to solubilize a substantial portion of the agrochemical;
E. Solid agrochemicals (at ambient temperature) that may be adsorbed to the liquid-liquid interface between the continuous aqueous phase and the disperse oil phase, and thereby serve as colloidal solids to form the Pickering emulsion. Such solid agrochemicals have solubility at 20° C. of below about 100 mg/l in both oil and aqueous phases present in the formulation.

Water-insoluble agrochemicals suitable for use in the present invention can readily be determined by one skilled in the art. The physical properties of agrochemical, such as water solubility and melting point, necessary to determine the suitability of an active ingredient in the present invention are well known and can be found in available publications such as The Pesticide Manual—14^{th} Edition (and subsequent editions plus the e-Pesticide manual), available from the British Crop Protection Council or readily determined by one of ordinary skill.

Substantially water-insoluble pesticidally active ingredients suitable for use in the present invention include, but are not limited to, fungicides such as azoystrobin, chlorothalonil, cyprodinil, difenoconazole, fludioxonil, mandipropamid, picoxystrobin, propiconazole, pyraclostrobin, tebuconazole, thiabendazole and trifloxystrobin; herbicides such as acetochlor, alachlor, ametryn, amidosulfuron, anilofos, atrazine, azafenidin, azimsulfuron, benfluralin, benfuresate, bensulfuron-methyl, bensulide, benzfendizone, benzofenap, bromobutide, bromofenoxim, bromoxynil, butachlor, butafenacil, butamifos, butralin, butylate, cafenstrole, carbetamide, chlorbromuron, chloridazon, chlorimuron-ethyl, chlorotoluron, chlorpropham, chlorthal-dimethyl, chlorthiamid, cinidon-ethyl, cinmethylin, cinosulfuron, clodinafop-propargyl, clomazone, clomeprop, cloransulam-methyl, cyanazine, cycloate, cyclosulfamuron, daimuron, desmedipham, desmetryn, dichlobenil, diflufenican, dimefuron, dimepiperate, dimethachlor, dimethametryn, dimethenamid, dimethenamid-P, dinitramine, dinoterb, diphenamid, dithiopyr, diuron, EPTC, esprocarb, ethalfluralin, ethametsulfuron-methyl, ethofumesate, etobenzanid, fenoxaprop-ethyl, fenoxaprop-P-ethyl, fentrazamide, fenuron, flamprop-methyl, flamprop-M-isopropyl, flazasulfuron, fluazolate, fluchloralin, flufenacet, flumiclorac-pentyl, flumioxazin, fluometuron, fluorochloridone, flupoxam, flurenol, fluridone, flurtamone, fluthiacet-methyl, halosulfuron-methyl, imazosulfuron, indanofan, isoproturon, isouron, isoxaben, isoxaflutole, lenacil, linuron, mefenacet, mesotrione, metamitron, metazachlor, methabenzthiazuron, methyldymron, metobenzuron, metobromuron, metolachlor, metosulam, metoxuron, metribuzin, metsulfuron-methyl, molinate, monolinuron, naproanilide, napropamide, neburon, norflurazon, orbencarb, oryzalin, oxadiargyl, oxadiazon, oxasulfuron, oxyfluorfen, pebulate, pendimethalin, pentanochlor, pethoxamid, pentoxazone, phenmedipham, pinoxaden, piperophos, pretilachlor, primisulfuron, prodiamine, profluazol, prometon, prometryn, propachlor, propanil, propazine, propham, propisochlor, propyzamide, prosulfocarb, prosulfuron, pyraflufen-ethyl, pyrazogyl, pyrazolynate, pyrazosulfuron-ethyl, pyrazoxyfen, pyributicarb, pyridate, pyriminobac-methyl, quinclorac, siduron, simazine, simetryn, S-metolachlor sulcotrione, sulfentrazone, sulfometuron-methyl, sulfosulfuron, tebutam, tebuthiuron, terbacil, terbumeton, terbuthylazine, terbutryn, thenylchlor, thiazopyr, thidiazimin, thifensulfuron-methyl, thiobencarb, tiocarbazil, triallate, triasulfuron, tribenuron-methyl, trietazine, trifluralin, triflusulfuron-methyl and vernolate; herbicide safeners such as benoxacor, cloquintocet, cloquintocet-mexyl, dichlormid, fenchlorazole-ethyl, fenclorim, flurazole, fluxofenim, furilazole, isoxadifen-ethyl, mefenpyr; alkali metal, alkaline earth metal, sulfonium or ammonium cation of mefenpyr; mefenpyr-diethyl and oxabetrinil; insecticides such as abamectin, clothianidin, emamectin benzoate, gamma cyhalothrin, imidacloprid, lambda cyhalothrin, permethrin, resmethrin and thiamethoxam.

Preferred substantially water-insoluble pesticidally active ingredients include acetamide herbicides and safeners. Representative acetamide herbicides include diphenamid, napropamide, naproanilide, acetochlor, alachlor, butachlor, dimethachlor, dimethenamid, dimethenamid-P, fentrazamide, metazachlor, metolachlor, pethoxamid, pretilachlor, propachlor, propisochlor, S-metolachlor, thenylchlor, flufenacet and mefenacet. Where the acetamide herbicide is liquid at ambient temperatures, i.e., has a melting point below about 0° C., the oil phase can consist essentially or substantially of the acetamide herbicide itself. In other words, no organic solvent is necessary, although one can optionally be included. Examples of acetamide herbicides that are liquid at ambient temperatures and can be formulated in compositions of the invention without the need for an organic solvent include acetochlor, butachlor, dimethenamid, dimethenamid-P, metolachlor, S-metolachlor and pretilachlor. Where an organic solvent is desired or required, any suitable organic solvent known in the agricultural chemical formulating art in which the acetamide herbicide is adequately soluble can be used. Preferably the organic solvent is one in which the acetamide herbicide is highly soluble, so that as high as possible a concentration of the acetamide herbicide can be accommodated in the oil phase and in the composition as a whole.

As used herein, the term acetamide includes mixtures of the two or more acetamides as well as mixtures of optical isomers of the acetamides. For example, mixtures of the (R) and (S) isomers of metolachlor wherein the ratio of (S)-2-chloro-N-(2-ethyl-6-methylphenyl)-N-(2-methoxy-1-methylethyl)acetamide to (R)-2-chloro-N-(2-ethyl-6-methylphenyl)-N-(2-methoxy-1-methylethyl)acetamide is in the range of from 50-100% to 50-0%, preferably 70-100% to 30-0% and more preferably 80-100% to 20-0% are included.

Preferred acetamides include mixtures of metolachlor (S) and (R) isomers wherein the ratio of (S)-2-chloro-N-(2-ethyl-6-methylphenyl)-N-(2-methoxy-1-methylethyl)acetamide to (R)-2-chloro-N-(2-ethyl-6-methylphenyl)-N-(2-methoxy-1-methylethyl)acetamide is in the range of from 50-100% to 50-0%, preferably 70-100% to 30-0% and more preferably 80-100% to 20-0%.

Safeners suitable for use in the present invention include benoxacor; cloquintocet; cloquintocet-mexyl; dichlormid; fenchlorazole-ethyl; fenclorim; flurazole; fluxofenim; furilazole; isoxadifen-ethyl; mefenpyr; an alkali metal, alkaline earth metal, sulfonium or ammonium cation of mefenpyr; mefenpyr-diethyl and oxabetrinil. Preferred safeners include benoxacor and dichlormid. When a liquid acetamide is used the safener will generally be dissolved in the acetamide phase. However, an organic solvent can optionally be used. Where an organic solvent is desired or required, any suitable organic solvent known in the agricultural chemical formulating art in which the acetamide herbicide and safener are adequately soluble can be used. Preferably the organic solvent is one in which the acetamide herbicide and safener are highly soluble, so that as high as possible a concentration of the active components can be accommodated in the oil phase and in the composition as a whole.

The same active compounds can be incorporated into the solid lipid stabilising particles. For example, particles of palmitin or similar are formed by dispersion of the melted palmitin into water or a surfactant solution. The palmitin will have some active ingredient dissolved or dispersed into it whilst melted. Thus a dispersion of particles of palmitin containing active ingredient is produced to then be employed as the solid lipid colloid stabilizer.

In one embodiment, the colloidal solids have a median particle size diameter as measured by suitable sizing methodology such as light scattering of 0.5 micron or less, preferably 0.1 micron or less.

The type and amount of colloidal solid is selected so as to provide acceptable physical stability of the composition. This can readily be determined by one of skill in the art by routine evaluation of a range of compositions having different amounts of these components. Typically, physical stability of the composition is acceptable if no significant coalescence is evident following storage for at least 7 days over the range of temperatures from 0° C. to about 50° C. Stable compositions within the scope of the present invention also include those compositions which can easily be re-suspended or re-dispersed with only a minor amount of agitation.

In one embodiment, when the continuous phase is aqueous, the continuous phase of the liquid agrochemical emulsion compositions comprises at least one water-soluble agrochemical. Preferably, the water-soluble agrochemical is an agrochemical electrolyte.

The water-soluble agrochemical electrolyte may be a pesticidally active ingredient or an adjuvant (enhancer) such as ammonium sulfate or any other ionic species added to a chemical formulation. The term “agrochemical” includes compounds which possess biological activity, for example herbicides, plant growth regulators, algicides, fungicides, bactericides, viricides, insecticides, acaricides, nematicides or molluscicides. Suitable agrochemicals which are water-soluble include acifluorfen, acrolein, aminopyralid, amitrole, asulam, benazolin, bentazone, bialaphos, bromacil, bromoxynil potassium, chloramben, chloroacetic acid, clopyralid, 2,4-D, 2,4-DB, dalapon, dicamba, dichlorprop difenzoquat, diquat, endothall, fenac, fenoxaprop, flamprop, flumiclorac, fluoroglycofen, flupropanate, fomesafen, fosamine, glufosinate, glyphosate, imidazolinones such as imazameth, imazamethabenz, imazamox, imazapic, imazapyr, imazaquin and imazethapyr, ioxynil, MCPA, MCPB, mecoprop, methylarsonic acid, naptalam, nonanoic acid, paraquat, picloram, quinclorac, sulfamic acid, 2,3,6-TBA, triclopyr and water-soluble salts thereof. Preferred agrochemicals include glyphosate (N-phosphonomethylglycine), which is commonly used in the form of its water-soluble salts such as potassium, trimethylsulphonium, isopropylamine, sodium, or ammonium salts, salts of diquat, for example diquat dibromide, fomesafen which is commonly used in the form of its water-soluble sodium salt, glufosinate which is commonly used in the form of its water-soluble ammonium salt, paraquat dichloride, dicamba which is commonly used in the form if its sodium or potassium or dimethlyammonium salts, and bentazone which is commonly used in the form of its water-soluble sodium salt. Representative agrochemical enhancers include ammonium nitrate, ammonium sulfate, sodium chloride and sodium acetate. While these components, alone, may not be pesticidally active they may be present to enhance the biological efficacy of the pesticide, to reduce the corrosion potential, to lower the freezing point, and/or to enhance the physical stability of the compositions. Thus for example glyphosate salts may be formulated or tank-mixed with ammonium sulfate as an activity enhancer, whilst magnesium sulfate may be added to paraquat as a purgative. Mixtures of water-soluble agrochemical electrolytes may also be used. Preferred mixtures include mixtures of glyphosate salts with at least one member selected from the group consisting of dicamba, diquat, glufosinate and paraquat.

The term “water-soluble” in relation to a pesticide or plant growth regulator or a salt thereof as used herein means having a solubility in deionized water at 20° C. sufficient to enable the water-soluble agrochemical electrolyte to be dissolved completely in the aqueous phase of a composition of the invention at the desired concentration. Preferred water-soluble agrochemicals useful in the present invention have a solubility in deionized water at 20° C. of not less than about 50,000 mg/l, more preferably not less than about 100,000 mg/l. Where an active compound is referred to herein as being water-soluble, but the compound itself is known not to be water-soluble as defined immediately above, it will be understood that the reference applies to water-soluble derivatives, more particularly water-soluble salts, of the compound.

The water-soluble agrochemical electrolyte, for example a herbicide, when present is at a concentration in the composition as a whole sufficient, upon dilution of the composition in a suitable volume of water, if required, and applied by spraying to the target locus, to be pesticidally, for example herbicidally, effective. In a concentrate composition it is desirable to provide as high a concentration, or “loading”, of the water-soluble active ingredient as is possible and convenient. Depending on the active compound in question and the intended use of the composition, a loading of about 50,000 to about 560,000 mg/l or higher is preferred.

Preferably, the water-soluble agrochemical electrolyte comprises at least one member selected from the group consisting of ammonium sulfate, magnesium sulfate, dicamba, diquat, glufosinate, glyphosate, paraquat and agriculturally acceptable salts thereof. In a particular embodiment, the water-soluble agrochemical electrolyte comprises an agriculturally acceptable salt of the herbicide glyphosate.

In one aspect of the present invention, an active compound (2) is contained within particles such as solid lipid particles.

In another aspect of the present invention, the continuous phase comprises an active compound (3).

In further aspects of the present invention, active compounds (1), (2) and (3) are independently each an agrochemical, a pharmaceutical, a food additive, a cleaning agent or a personal care substance; preferably active compounds (1), (2) and (3) are independently each an agrochemical. Active compounds (1), (2) and (3) may be the same or different.

An active compound may be a pharmaceutical agent. Examples of pharmaceutical agents include nucleic acids, proteins and peptides, hormones and steroids, chemotherapeutics, NSAIDs, vaccine components, analgesics, antibiotics and anti-depressants. It may be desirable to provide sustained release of one or more pharmaceutical agents.

An active compound may be a food additive. Examples of food additives include flavourants and dietary supplements including amino acids, vitamins, minerals, anti-oxidants, prebiotics and herbal extracts.

Examples of substances (actives) that may be encapsulated for different functionalities include:
Flavours (both taste and aroma components)—eg quinine, citric acid, sucrose
Antioxidants: polyphenols (eg gallic acid; catechins such as epigallocatechin-3-gallate); essential fatty acids (eg omega-3 and omega-6)
Nutrients: polysaccharides (eg celluloses, starches, carrageenans), simple sugars (eg glucose), proteins (eg gelatin), natural oils (eg sunflower, olive), natural fats (eg triglycerides)
Micronutrients: vitamins (eg A, B, C, D and E), Minerals (eg sodium, potassium)

More specific examples in the food and drink area include quinine and caffeine. Pharmaceuticals include paracetamol and prednisolone.

An active compound may be a cleaning agent including surfactants, silicones and sanitizing agents such as antimicrobial agents and alcohols.

An active compound may be a personal care substance. Examples include fragrances, skin-care additives, botanicals, astringents, moisturisers and emollients and lubricants.

Lipids are compound that is virtually insoluble in water but can be soluble in organic non-polar solvents (hydrocarbons, chloroform, benzene, ether, alcohol etc.). Natural lipids are large and diverse group of compounds that are biodegradable and non-toxic and include the following:
1. Fatty acids
   a. saturated
   b. unsaturated
2. Glycerides
   a. Neutral glycerides
   b. Phosphoglycerides
3. Complex lipids (lipoproteins)
4. Non-glyceride lipids
   a. Waxes
   b. Steroids
   c. Sphingolipids

Lipid particles can be produced from lipids that are solid at the storage temperatures required for the product. Since the storage temperature requirements will differ for different product concepts and utilities, a simple method such as DSC (Differential Scanning calorimetry) can determine whether the chosen solid lipid will have suitable characteristics for the chosen use (examples include fatty acids and acylglycerols). Fatty acids are long-chain (10-30 carbon atoms) monocarboxylic acid compounds and may be saturated (e.g. lauric acid, myristic acid, palmitic acid, capric acid, staric acid, arachidic acid etc.) or unsaturated (palmitoleic acid, oleic acid, linoleic acid, linolenic acid, arachidonic acid etc). Unsaturated fatty acids have lower melting points than saturated fatty acids.

Acylglycerols are the most common class of lipids and consists of fatty acids linked with the trihydric alcohol (glycerol) via an ester bond. Depending on the mole ratio of glycerol to fatty acid acylglycerols can be divided to monoacylglycerols (1 mole glycerol 1 mole fatty acid, e.g. monopalmitin), diacylglycerols (1 mole glycerol 2 mole fatty acid, e.g. dipalmitin) and triacylglycerols (1 mole glycerol 3 mole fatty acid, e.g. tripalmitin). Triacylglycerols having identical acyl chains are termed “simple” (e.g. tristearin, tripalmitin) and those having different acyl chains are termed “mixed” (e.g. 1-stearo dipalmitin, 1-palmito distearin, 2-stearo dipalmitin, 2-palmito distearin).

Waxes are type of neutral lipids and most commonly are composed of a long-chain (typically more than 12 carbon atoms) monohydric alcohol and long chain fatty acids linked together with an ester oxygen. Typical animal/vegetable waxes are selected from beeswax, Carnauba wax, Jojoba wax, spermaceti, lanolin, tallow, Candelilla wax and soy wax.

Other types of waxes include:
1. Petroleum derived (e.g. paraffin wax, microcrystalline wax)
2. Mineral derived (lignite, peat, Ozocerite)
3. Synthetic (e.g. fatty acid amide wax, polyolefin wax, Fischer-Tropsch wax, polar synthetic wax).

The emulsions of the present invention may be prepared via the following process steps:
1. A lipid (solid at room temperature) is melted.
2. If required, an active compound is added to molten lipid.
3. The molten lipid is emulsified into water or an aqueous surfactant solution at a temperature above the melting point of the lipid to form an emulsion. Surfactant may alternatively be added to the molten lipid mix dependent on the surfactant type and processing convention.
4. The emulsion is then cooled to solidify the lipid droplets to form a suspension of solid lipid particles in an aqueous phase.
5. The suspension may be dialysed in dialysis tubing against fresh water to remove any excess surfactant from the suspension.

The suspension prepared above may then be used to prepare O/W or W/O Pickering emulsions in the following manners:

### O/W Emulsions

6a. An oil phase is emulsified into the lipid suspension to form a Pickering emulsion of oil droplets in water stabilized by discrete lipid particles at the o/w interface. The oil phase may be a single oil or a solution of an active compound in a suitable oil or an adjuvant oil. In addition, the oil may also have dissolved within it a monomer or pre-polymer.
7a. The interface may be further modified by either warming to sinter the particles into a continuous layer or into a layer of connected (fused) particles. With the latter, a crosslinking agent may be added to the aqueous phase to react with any monomer or pre-polymer included in the oil phase to form a polymeric layer that either partially or completely surrounds the particles or fills the interstices between the particles.

### W/O Emulsions

6b. The lipid suspension is emulsified into an oil phase to form a Pickering emulsion of aqueous droplets in oil stabilized by discrete lipid particles at the o/w interface. The lipid suspension may be further modified by adding to it a crosslinking agent.
7b. The interface may be further modified by either warming to sinter the particles into a continuous layer or into a layer of connected (fused) particles. With the latter, a monomer or pre-polymer may be added to the oil phase to react with any crosslinking agent included in the lipid suspension to form a polymeric layer that either partially or completely surrounds the particles or fills the interstices between the particles.
8. Optional additional compounds may be added to the continuous phase.

In a further aspect then, the present invention provides an emulsion where the colloidal solid lipid particles are present as discrete particles. These discrete particles may be either partially or fully surrounded by a polymer which is also located at the interface between the continuous phase and the dispersed phase (the polymer may be immediately above the colloidal particles or immediately below the particles or situated between the particles); so the polymer may be located in a variety of ways such that the particles reside on a layer of polymer; the particles are overlaid by polymer; the polymer fills gaps (interstices) between the particles; or a combination of these ways.

In an alternative aspect, the present invention provides an emulsion where the colloidal solid lipid particles are present as fused (or sintered) particles.

The following concentrations, by weight, are preferred:

In the lipid emulsion/suspension: 0-20% lipid; 0-10% surfactant; 0-0.1% preservative.

The concentration of dissolved active compound in the lipid depends on solubility, but it is suitably 0-50%. Any monomer or pre-polymer is present at 0-10%. Any dissolved active compound in the aqueous phase of the suspension may be present at 0-50%. Any cross-linker may be present at 0-5% (ratio to the polymer). In a similar manner, for a water-in-oil emulsion, any dissolved active in the continuous oil phase of the suspension may be present at 0-50%.

In the final emulsion of the present invention the concentrations in the different phases can be varied within wide limits. Typical emulsions can contain up to 50% dispersed phase, so that an active dissolved in the dispersed phase could constitute up to 25% of the total product. Actives can be dissolved in both the dispersed and continuous phases. In the special case where an active is an oil, the active can therefore constitute up to 50% of a phase. Solid lipid can be present at up to 20% of the solid lipid dispersion; if the solid lipid dispersion is employed at 20% of the product in a W/O emulsion (see examples later) an active contained within the lipid can be available the product at up to 2%. In the similar O/W emulsion where the lipid particle dispersion can be employed at up to 80% of the product (see examples later), the active contained within the lipid particles can constitute up to 8% of the product.

The invention is illustrated by way of reference to the Figures. Throughout this document, all quantities are expressed as w/w % unless otherwise stated.

The emulsions may be used to develop active ingredients to, for example, the human body, such as the skin, mouth or selectively to parts of the gastrointestinal tract. The emulsions may be adapted to selectively deliver the ingredients, such as in a topical formulation or oral administered formulation.

FIG. 1 shows a Cryo-SEM image of tripalmitin particles with no emulsifier.

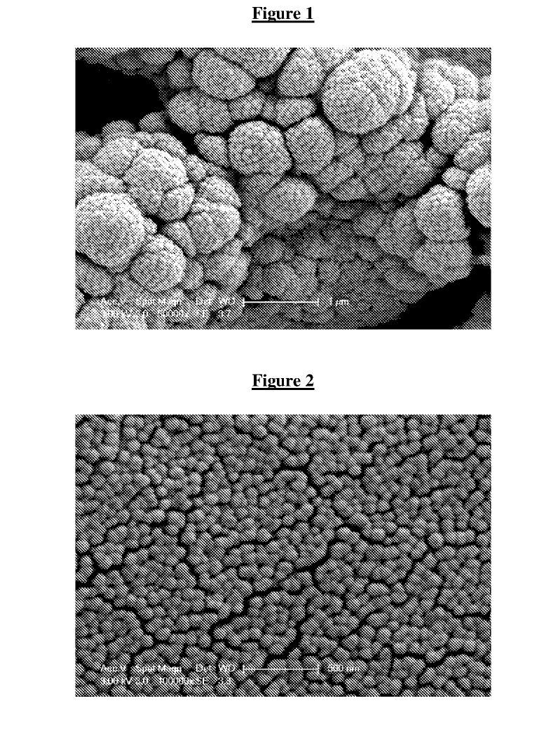

FIG. 2 shows a Cryo-SEM image of tripalmitin particles with Tween™ 20.

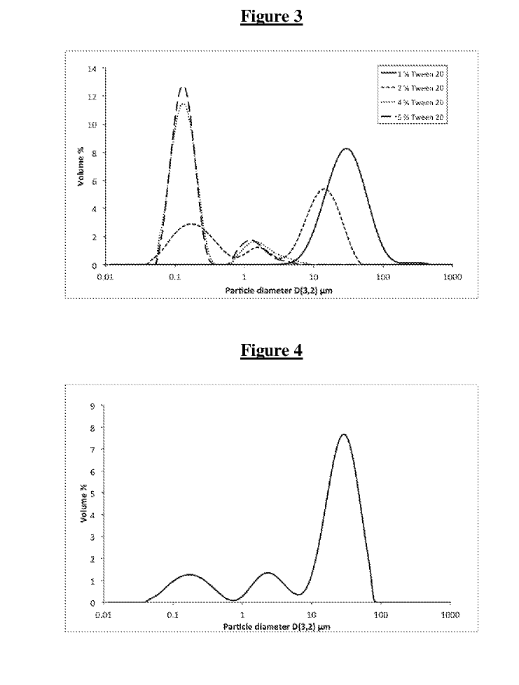

FIG. 3 shows the reduction in tripalmitin particle size by the addition of 1% to 5% w/w Tween™ 20.

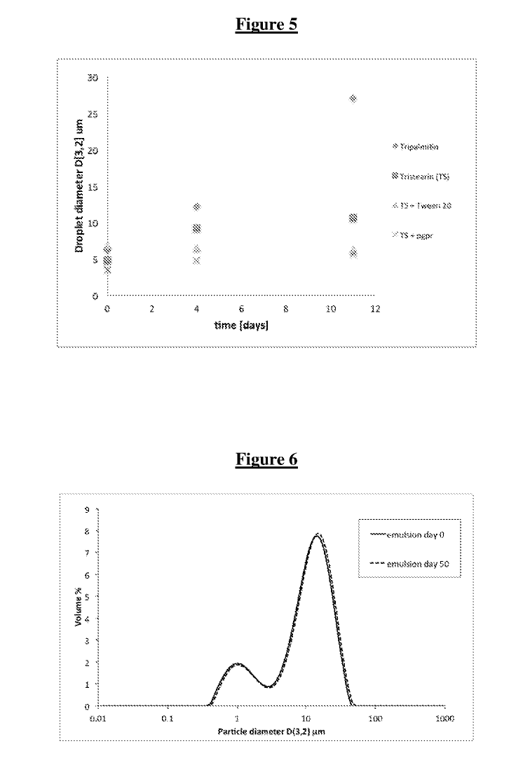

FIG. 4 shows multimodal size distributions of tripalmitin without emulsifier.

FIG. 5 shows droplet size of lipid-particles-stabilised-w/o emulsion stabilised without emulsifier or with PGPR or Tween™ 20.

FIG. 6 shows sunflower oil in water emulsions stabilised with tripalmitin particles are stable over an observation period of 50 days.

FIG. 7 shows release pf lipophilic active (Sudan III).

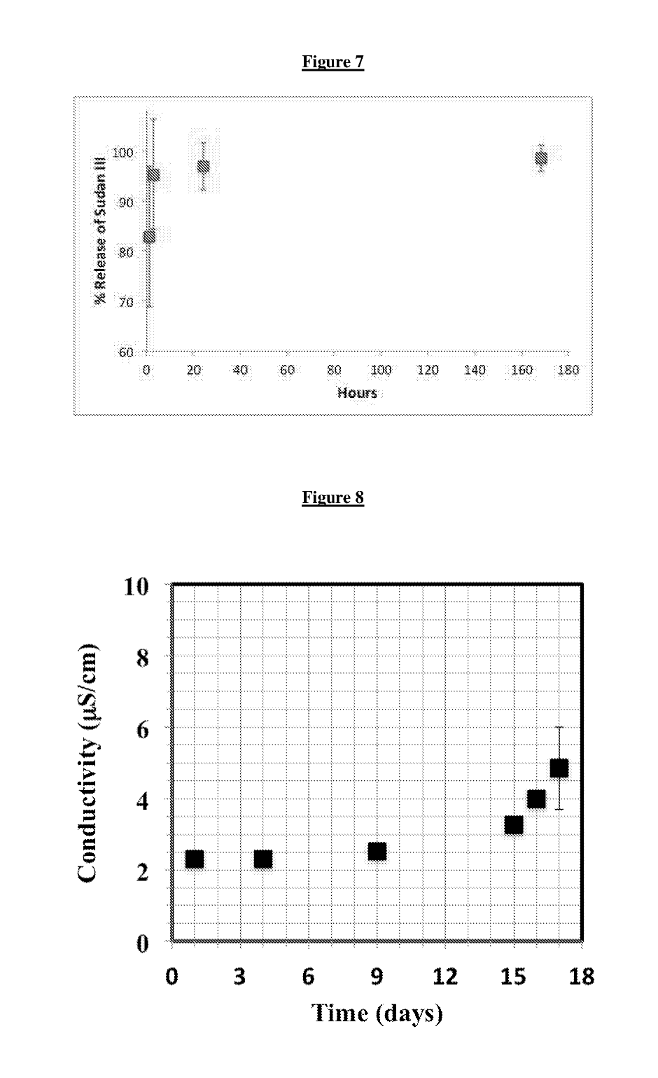

FIG. 8 shows that the release of hypophilic active (NaCl) over 17 days is small.

FIG. 9 shows the conductivity of aqueous phase of NaCl released from sunflower oil, silicon oil and mineral oil.

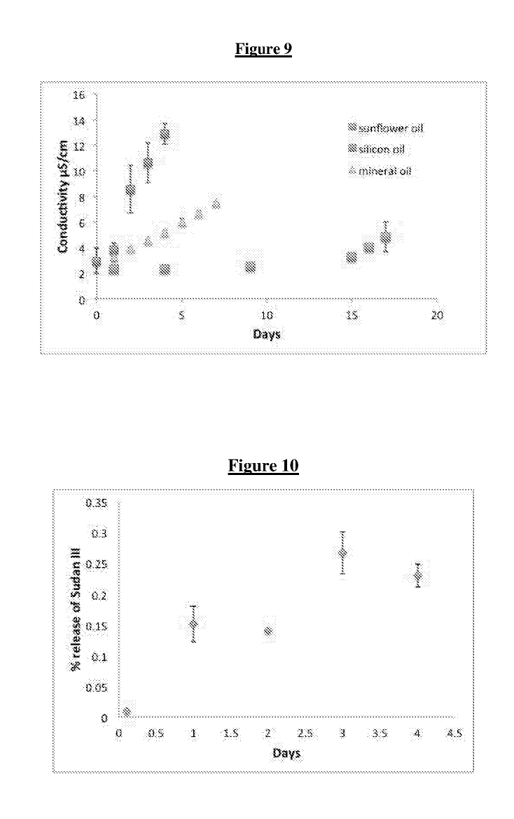

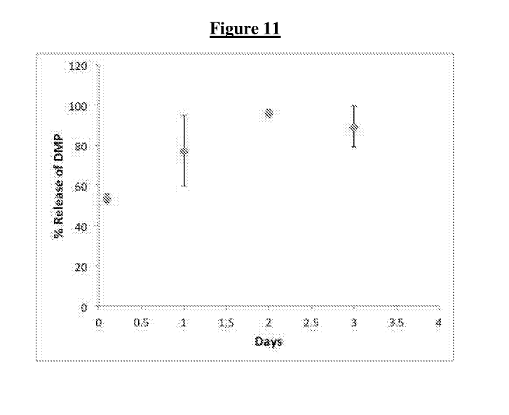

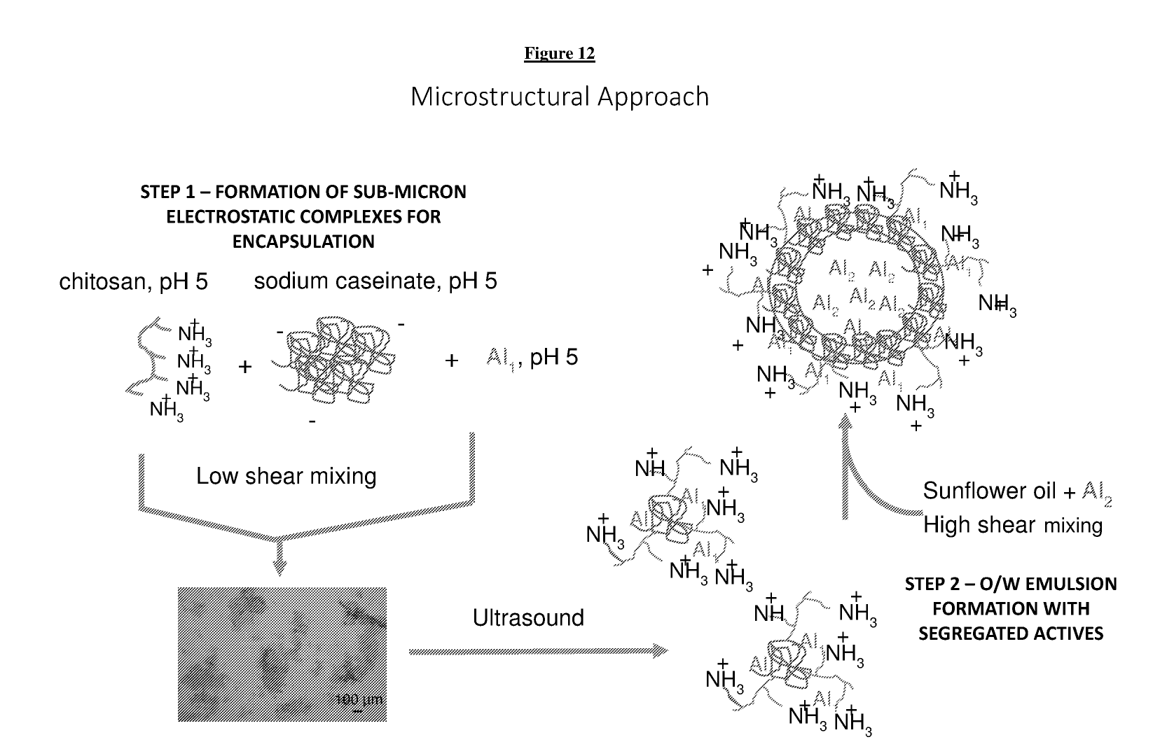

FIG. 10 shows the release of Sudan III from lipid particles stabilised in aqueous dispersion by WPI.

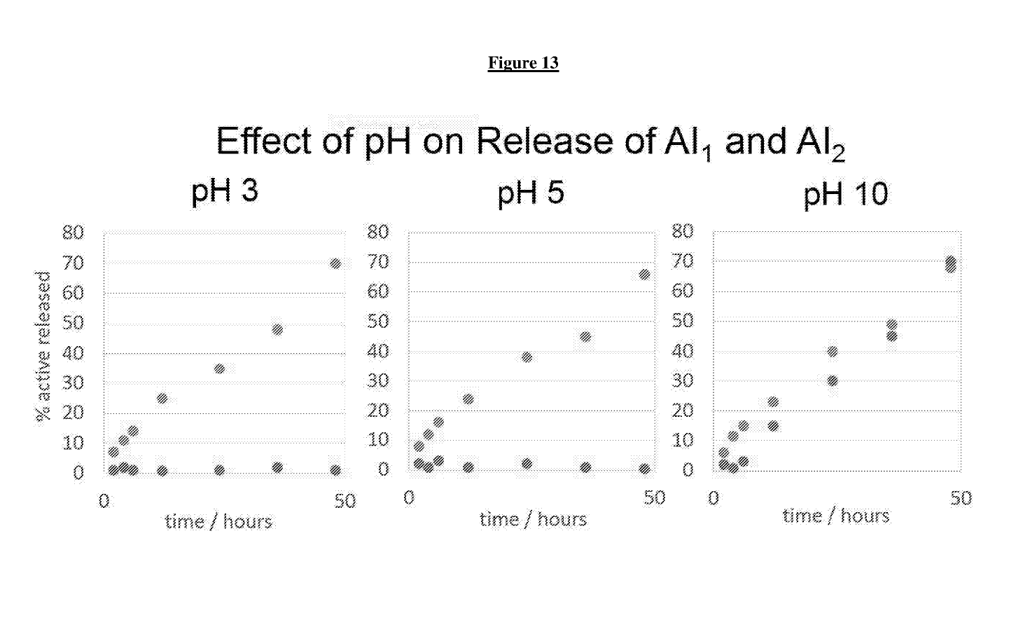

FIG. 11 shows the release of DMP (dimethyl phthalate) from o/w emulsion.

FIG. 12 is a schematic drawing showing the production of emulsions from chitosan, sodium caseinate, fluorescein, sunflower oil and rhodamine.

FIG. 13 shows the differential release of fluorescein and rhodamine of the system shown in FIG. 12 at different pH.

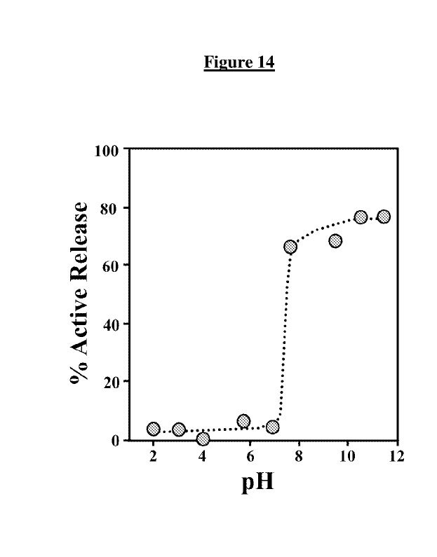

FIG. 14 shows pH-triggered release for protein-polysaccharide complexes.

FIG. 15 shows droplet size evolution of simple o/w emulsions stabilised by functional Pickering particles (polysaccharide/protein complexes). Depending on the polysaccharide/protein mass ration, long-term emulsion stability (up to two months following emulsion preparation) can be achieved by using either active-containing or non active-containing polysaccharide/protein complexes.

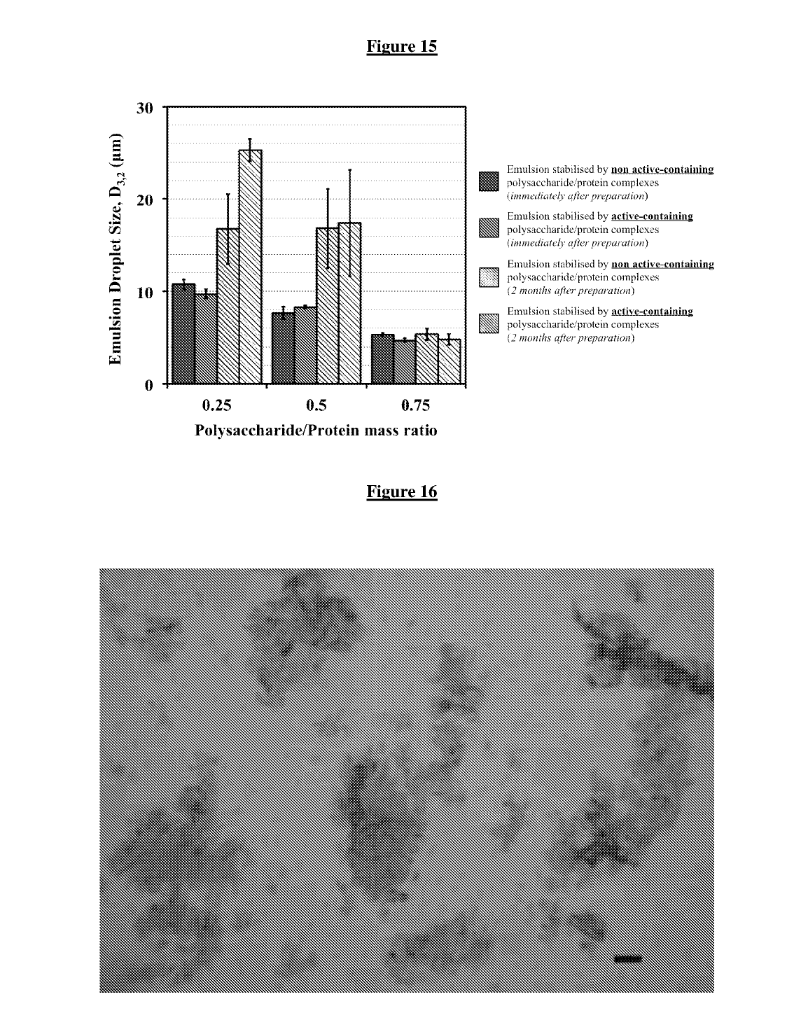

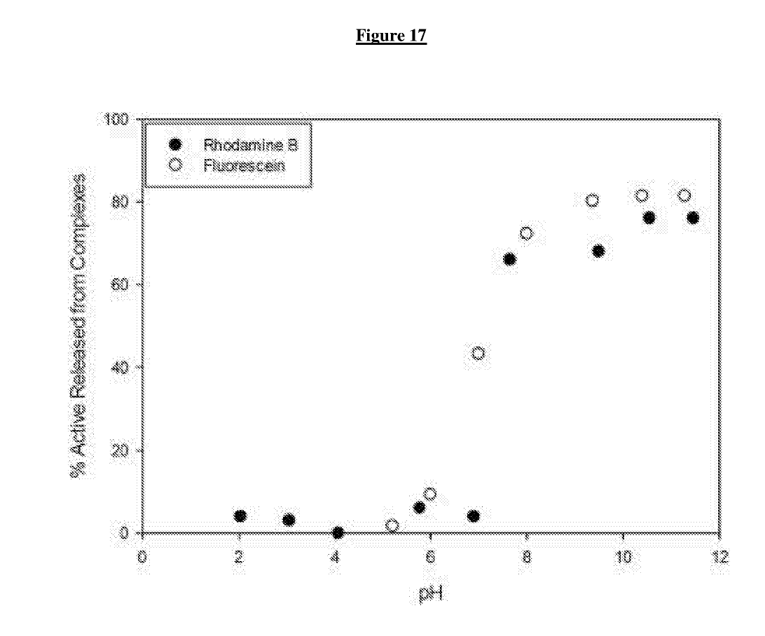

FIG. 16 Shows a representative optical micrograph showing morphology of NaCAS-chitosan complexes formed under low shear. Scale bar=100 micron.

FIG. 17 shows a release of fluorescein (FSS) and Rhodamine B (Rhod B) from the fabricated complexes as a function of pH.

### EXAMPLE 1

This series of examples illustrates the preparation of an aqueous dispersion of solid lipid particles, which can then be employed as colloid stabilisers in the preparation of an emulsion.

3 g of a solid triacylglycerol (e.g. tripalmitin, tristearin, 1:1, 2:1 and 1:2 mixture of tristearin and tripalmitin, 2:1 mixture of tristearin and monopalmitin, 1:1 mixture of tristearin and Carnauba solid lipid; weight ratios) was combined with 0-3 g emulsifier (e.g. Tween™ 20, Whey Protein Isolate=WPI, Lecithin, 1:1 mixture of Lecithin and Tween 20, Polyglycerol Polyricinoleate=PGPR), 0.006 g preserving agent (potassium sorbate) and 53.994-56.994 g double distilled water in a 100 ml glass beaker. The formulations are shown in Table 1.

A total 60 g batch mixture was heated to 75-80° C. (temperature above the melting point of the lipids and below the cloud point of Tween™ 20) while stirred with a magnetic stirrer for 10 min. Subsequently the hot mixture was sonicated for 3 minutes at constant amplitude of 95%, using a sonicating probe. Immediately after sonication, the batch was cooled in an ice bath for up to 30 minutes and then stored at 4-8° C.

Cryo-SEM images of (etched) samples of tripalmitin particles with no emulsifier and with Tween™ 20 are shown in FIG. 1 and FIG. 2, respectively.

The particle size of solid particles in water can be modified by the type and the amount of the emulsifier used to stabilise those particles. For example, the reduction in tripalmitin particle size (from ˜25 μm to 150 nm) could be obtained by an increase in the concentration of Tween™ 20 (from 1% to 5%), as shown in FIG. 3. The particles produced with no emulsifier have multimodal size distribution shown in FIG. 4. Further reduction in the particle size may be obtained by: (i) application of higher hear during their production (e.g. high-pressure homogeniser, micro-fluidiser) and/or (ii) using emulsifiers that allow faster decrease in interfacial tension (e.g. sodium dodecyl sulphate).

TABLE 1

| | Concentration (w/w %) | | | | | | | | | |
|---|---|---|---|---|---|---|---|---|---|---|
| Ingredient | 1A | 1B | 1C | 1D | 1E | 1F | 1G | 1H | 1I | 1J |
| Tripalmitin | 5 | | 5 | 5 | 5 | 5 | 5 | | 2.5 | 3.33 |
| Tristearin | | 5 | | | | | | 5 | 2.5 | 1.67 |
| Monopalmitin | | | | | | | | | | |
| Carnauba | | | | | | | | | | |
| wax | | | | | | | | | | |
| Tween ™ 20 | | | 1 | 2 | 3 | 4 | 5 | 5 | 5 | 5 |
| WPI | | | | | | | | | | |
| Lecithin | | | | | | | | | | |
| PGPR | | | | | | | | | | |
| Potassium | 0.01 | 0.01 | 0.01 | 0.01 | 0.01 | 0.01 | 0.01 | 0.01 | 0.01 | 0.01 |
| sorbate | | | | | | | | | | |
| Distilled | 94.99 | 94.99 | 93.99 | 92.99 | 91.99 | 90.99 | 89.99 | 89.99 | 89.99 | 89.99 |
| water | | | | | | | | | | |
| Total | 100 | 100 | 100 | 100 | 100 | 100 | 100 | 100 | 100 | 100 |

| | Concentration (w/w %) | | | | | | | | |
|---|---|---|---|---|---|---|---|---|---|
| Ingredient | 1K | 1L | 1M | 1M | 1O | 1P | 1R | 1Z | 1X |
| Tripalmitin | 1.67 | | | 5 | | 5 | | 5 | |
| Tristearin | 3.33 | 3.33 | 3.33 | | 5 | | 5 | | 5 |
| Monopalmitin | | 1.67 | | | | | | | |
| Carnauba | | | 1.67 | | | | | | |
| wax | | | | | | | | | |
| Tween ™ 20 | 5 | 5 | 5 | | | 2.5 | 2.5 | | |
| WPI | | | | 5 | 5 | | | | |
| Lecithin | | | | | | 2.5 | 2.5 | | |
| PGPR | | | | | | | | 1 | 1 |
| Potassium | 0.01 | 0.01 | 0.01 | 0.01 | 0.01 | 0.01 | 0.01 | 0.01 | 0.01 |
| sorbate | | | | | | | | | |
| Distilled | 89.99 | 89.99 | 89.99 | 89.99 | 89.99 | 89.99 | 89.99 | 93.99 | 93.99 |
| water | | | | | | | | | |
| Total | 100 | 100 | 100 | 100 | 100 | 100 | 100 | 100 | 100 |

### EXAMPLE 2

This series of examples illustrates the preparation of an aqueous dispersion of lipid particles containing lipophilic model active encapsulated within the lipid matrix that can then be employed as a colloid stabilizer in the preparation of an emulsion.

3 g of a solid triacylglycerol (e.g. tripalmitin, tristearin, 1:1, 2:1 and 1:2 mixture of tristearin and tripalmitin, 2:1 mixture of tristearin and monopalmitin, 1:1 mixture of tristearin and Carnauba solid lipid) was combined with 0.03 g model lipophilic active (Sudan III) while heated above the lipid melting point and stirred with the magnetic stirrer until the active dissolved (ca. 0.5 hour). This was then combined with 0-3 g emulsifier (e.g. Tween™ 20, WPI=Whey Protein Isolate, Lecithin, 1:1 mixture of Lecithin and Tween™ 20, PGPR=Polyglycerol Polyricinoleate), 0.006 g preserving agent (potassium sorbate) and 53.964-56.964 g double distilled water in a 100 ml glass beaker. The formulations are shown in Table 2.

The total 60 g batch mixture was heated to 75-85° C. (temperature above the melting point of the lipids and below the cloud point of Tween™ 20) while stirred with the magnetic stirrer for 10 minutes. Then the hot mixture was sonicated for 3 min at constant amplitude of 95% using sonicating probe. Immediately after sonication, the batch was cooled in an ice bath for up to 30 minutes and then stored at 4-8° C.

Dialysis of the excess surfactant. When Tween™ 20 was used to stabilise lipid particles, the colloidal suspensions were placed in pre-hydrated cellulose tubing (12 kD cut off) and dialysed into distilled water. The dialysis water was changed numerous times until the measured surface tension reached a constant value.

The particle size of solid lipid particles with the model active compound were of similar range as the particles without the model active (shown in Example 1).

TABLE 2

| | Concentration (w/w %) | | | | | | | |
|---|---|---|---|---|---|---|---|---|
| Ingredient | 2A | 2B | 2C | 2D | 2E | 2F | 2G | 2H |
| Tripalmitin | 5 | | 5 | | 2.5 | 3.33 | 1.67 | |
| Tristearin | | 5 | | 5 | 2.5 | 1.67 | 3.33 | 3.33 |
| Monopalmitin | | | | | | | | 1.67 |
| Carnauba | | | | | | | | |
| wax | | | | | | | | |
| Tween ™ 20 | | | 5 | 5 | 5 | 5 | 5 | 5 |
| WPI | | | | | | | | |
| Lecithin | | | | | | | | |
| PGPR | | | | | | | | |
| Lipophilic | 0.05 | 0.05 | 0.05 | 0.05 | 0.05 | 0.05 | 0.05 | 0.05 |
| active (Sudan | | | | | | | | |
| III) | | | | | | | | |
| Potassium | 0.01 | 0.01 | 0.01 | 0.01 | 0.01 | 0.01 | 0.01 | 0.01 |
| sorbate | | | | | | | | |
| Distilled H₂O | 94.94 | 94.94 | 89.94 | 89.94 | 89.94 | 89.94 | 89.94 | 89.94 |
| Total | 100 | 100 | 100 | 100 | 100 | 100 | 100 | 100 |

| | Concentration (w/w %) | | | | | | |
|---|---|---|---|---|---|---|---|
| Ingredient | 2I | 2J | 2K | 2L | 2M | 2N | 2O |
| Tripalmitin | | 5 | | 5 | | 5 | |
| Tristearin | 3.33 | | 5 | | 5 | | 5 |
| Monopalmitin | | | | | | | |
| Carnauba | 1.67 | | | | | | |
| wax | | | | | | | |
| Tween ™ 20 | 5 | | | 2.5 | 2.5 | | |
| WPI | | 5 | 5 | | | | |
| Lecithin | | | | 2.5 | 2.5 | | |
| PGPR | | | | | | 1 | 1 |
| Lipophilic | 0.05 | 0.05 | 0.05 | 0.05 | 0.05 | 0.05 | 0.05 |
| active (Sudan | | | | | | | |
| III) | | | | | | | |
| Potassium | 0.01 | 0.01 | 0.01 | 0.01 | 0.01 | 0.01 | 0.01 |
| sorbate | | | | | | | |
| Distilled H₂O | 89.94 | 89.94 | 89.94 | 89.94 | 89.94 | 93.94 | 93.94 |
| Total | 100 | 100 | 100 | 100 | 100 | 100 | 100 |

### EXAMPLE 3

This example illustrates the preparation of a lipid-particles-stabilised-W/O-emulsion with a lipophilic active encapsulated within the particles and a hydrophilic active encapsulated within the dispersed phase.

10 g of aqueous dispersion of lipid particles (stabilised with no emulsifier or Tween™ 20 or PGPR or Lecithin or 1:1 mixture of Lecithin and Tween 20) containing lipophilic active (Sudan III, as per Example 2) were combined with 1 g of hydrophilic active (10% solution of NaCl) and 39 g of liquid oil (e.g. sunflower oil, mineral oil, silicone oil, methyl oleate, hexadecane) and homogenised with a rotor-stator mixer (Silverson™) for 1-3 minutes at 10,000 rpm, while cooled in an ice bath. Such prepared W/O emulsions were stored at 4-8° C. The formulation is shown in Table 3.

TABLE 3

| Ingre- | Concentration (w/w %) | | | | | | | | |
|---|---|---|---|---|---|---|---|---|---|
| dient | 3A | 3B | 3C | 3D | 3E | 3F | 3G | 3H | 3I |
| Lipid | 20 | | | | | | | | |
| particle | | | | | | | | | |
| dis- | | | | | | | | | |
| persion | | | | | | | | | |
| Exam- | | | | | | | | | |
| ple 2A | | | | | | | | | |
| Exam- | | 20 | | | | | | | |
| ple 2B | | | | | | | | | |
| Exam- | | | 20 | 20 | 20 | 20 | 20 | | |
| ple 2C | | | | | | | | | |
| Exam- | | | | | | | | 20 | |
| ple2D | | | | | | | | | |
| Exam- | | | | | | | | | 20 |
| ple 2O | | | | | | | | | |
| Sun- | 78 | 78 | 78 | | | | | 78 | 78 |
| flower | | | | | | | | | |
| oil | | | | | | | | | |
| Mineral | | | | 78 | | | | | |
| oil | | | | | | | | | |
| Silicone | | | | | 78 | | | | |
| oil | | | | | | | | | |
| Methyl | | | | | | 78 | | | |
| oleate | | | | | | | | | |
| Hexa- | | | | | | | 78 | | |
| decane | | | | | | | | | |
| Hydro- | 2 | 2 | 2 | 2 | 2 | 2 | 2 | 2 | 2 |
| philic | | | | | | | | | |
| active | | | | | | | | | |
| (10% | | | | | | | | | |
| NaCl | | | | | | | | | |
| solu- | | | | | | | | | |
| tion) | | | | | | | | | |
| Total | 100 | 100 | 100 | 100 | 100 | 100 | 100 | 100 | 100 |

The droplet size of obtained emulsions was lower than 10 μm and (except emulsions stabilised with tripalmitin particles) did not change significantly over the observation period of 11 days, as shown in FIG. 5.

### EXAMPLE 4

This example illustrates the preparation of a lipid-particles-stabilised-O/W-emulsion with two separated lipophilic actives (A encapsulated within the particles and B encapsulated within the dispersed phase).

40 g of the aqueous dispersion of lipid particles (stabilised with WPI) containing lipophilic active A (Sudan III, as per Example 2) were combined with 9.9 g of the oil phase (e.g. sunflower oil, silicone oil, mineral oil, methyl oleate, hexadecane) and 0.1 g of dimethyl phthalate (DMP) and homogenised with a rotor-stator mixer (Silverson™) for 1-3 minutes at 10,000 rpm while cooled in an ice bath. Such prepared O/W emulsions were stored at 4-8° C. The formulations are shown in Table 4.

TABLE 4

| | Concentration (w/w %) | | | | | |
|---|---|---|---|---|---|---|
| Ingredient | 4A | 4B | 4C | 4D | 4E | 4F |
| Lipid particle | 80 | | 80 | 80 | 80 | 80 |
| dispersion | | | | | | |
| Example 2J | | | | | | |
| Example 2K | | 80 | | | | |
| Sunflower oil | 19.8 | 19.8 | | | | |
| Mineral oil | | | 19.8 | | | |
| Silicone oil | | | | 19.8 | | |
| Methyl oleate | | | | | 19.8 | |
| Hexadecane | | | | | | 19.8 |
| Lipophilic active B | 0.2 | 0.2 | 0.2 | 0.2 | 0.2 | 0.2 |
| (DMP) | | | | | | |
| Total | 100 | 100 | 100 | 100 | 100 | 100 |

Sunflower oil-in-water emulsions stabilised with tripalmitin particles prepared with WPI have droplet size distribution (as shown in FIG. 6) and are stable over the observation period of 50 days.

### EXAMPLE 5

This example illustrates the release of both hydrophilic and lipophilic active ingredients from W/O emulsions.

Solid lipid particles were prepared according to Example 2C. Tripalmitin particles contained the lipophilic active (Sudan III) and were stabilised in the aqueous dispersion by Tween™ 20. After preparation the excess surfactant was dialysed off to distilled water.

W/O emulsions were prepared according to Example 3C. Hydrophilic active (NaCl) was added to the aqueous included phase and sunflower oil was used as the continuous phase.

### Release of Lipophilic Active (Sudan III)

Known quantity of the W/O emulsion (e.g. 45 g) was placed in the beaker and gently topped up with a known quantity of sunflower oil (e.g. 50 g) termed “external” oil phase. Aliquots (ca. 1 mL) of the external oil phase were taken at time intervals, pressed through a syringe filter (0.2 μm pore size) and analysed using UV-Vis spectroscopy (at λ=510 nm). Using previously obtained calibration line, the concentration of the released active was calculated.

### Release of Hydrophilic Active (NaCl)

Known quantity of the W/O emulsion (e.g. 45 g) was gently placed on the top of a known quantity of distilled water (e.g. 70 g) termed “external” water/aqueous phase. Calibrated conductivity probe was placed in the external aqueous phase and readings were taken on daily intervals.

### Results

The lipophilic active (Sudan III) is completely released during first 10 h of storage (i.e. burst release) as shown in FIG. 7.

The release of the hydrophilic active (NaCl) over 17 days is very small, as shown in FIG. 8. In case a total NaCl payload was released to the external water phase, the measurable conductivity would amount to ca. 1800 μS/cm, therefore for the clarity of the graph, absolute conductivity values were plotted rather than % release of NaCl. The small quantity of released NaCl is due to plasticising effect of sunflower oil, which enhances sintering of lipid particles at the interface, leading to better shielding of the encapsulated payload.

### EXAMPLE 6

The sintering of particles occurs at storage temperature of 4-8° C. due to plasticising effect of the oil phase used. The effect of non-sintered particles (when different oil phase was used) on the release of actives is shown in Example 7.

Upon heating to 40° C., the fat crystals at the interface melt, the emulsion becomes unstable and all dispersed water phase with NaCl is delivered to the external water phase.

### EXAMPLE 7

This example illustrates the effect of particle sintering on the release of the hydrophilic payload encapsulated within the dispersed phase of W/O emulsions.

Solid lipid particles were prepared according to Example 2C. Tripalmitin particles contained the lipophilic active (Sudan III) and were stabilised in the aqueous dispersion by Tween™ 20. After preparation the excess surfactant was dialysed off to distilled water.

W/O emulsions were prepared according to Example 3C, 3D and 3E. Hydrophilic active (NaCl) was added to the aqueous included phase and sunflower or silicone or mineral oils were used as the continuous phase.

The release of NaCl was measured as per Example 5.

### Results

The conductivity of aqueous phase is shown in FIG. 9. Total payload release results in the external water phase conductivity of ca. 1800 μS/cm, therefore for the clarity of the graph, absolute conductivity values were plotted rather than % release of NaCl.

Relatively high release of NaCl from water-in-silicone-oil-emulsions results from a very limited solid fat solubility in silicone oil. Therefore there is no sintering of particles at the interface and the internal water phase is not effectively “shielded”, which leads to ingredient migration to the external water phase.

Lower conductivity measured for samples of water-in-mineral-oil-emulsions suggest some plasticizing/sintering effect of mineral oil. Solid fat particles partially sinter at the interface leading to the release of the internal water phase with NaCl, which is lower than for the non-sintered silicone oil emulsions and higher than the sintered sunflower oil emulsions.

### EXAMPLE 8

This example illustrates the release of two segregated lipophilic actives A and B from O/W emulsion.

Solid lipid particles were prepared according to Example 2J. Tripalmitin matrix contained lipophilic active A (Sudan III) and were stabilised in aqueous dispersion by WPI.

O/W emulsions were prepared according to Example 4. A model lipophilic active B (DMP-dimethyl phthalate) was added to the sunflower oil included phase.

### Release of Lipophilic Active A (Sudan III)

Known quantity of the O/W emulsion (e.g. 40 g) was placed in the beaker and gently topped up with a known quantity of sunflower oil (e.g. 50 g), termed “external” oil phase. An aliquot (ca. 1 mL) of the external oil phase was taken at time intervals, pressed through a syringe filter (0.2 μm pore size) and analysed using UV-Vis spectroscopy (at λ=510 nm). Using previously obtained calibration line, the concentration of the released active A was calculated.

### Release of Lipophilic Active B (DMP)

Known quantity of the O/W emulsion (e.g. 10 g) was placed inside a pre-hydrated cellulose tube and dialysed to a known quantity of distilled water (e.g. 100 g), termed “external” water/aqueous phase. An aliquot (ca. 1 mL) of the external water was taken at time intervals, pressed through a syringe filter (0.2 μm pore size) and analysed using UV-Vis spectroscopy (at λ=290 nm). Using previously obtained calibration line and the partitioning (sunflower oil/water) data of DMP, the concentration of the released active B was calculated.

### Results

The release of active A (Sudan III) from the lipid particles is very low (below 1% over the observation period of 4 days) and shown in FIG. 10. The release of active B (DMP) from the dispersed sunflower oil phase is complete (100% released) after 2 days of storage, as shown in FIG. 11. Low release of Sudan III suggests that lipid particles stabilised with WPI are not easily plasticised by the sunflower oil dispersed phase of the emulsion. Absence of particles sintering (and so no “capsule” formation) is then responsible for fast release of active B (DMP) from the dispersed oil phase.

### EXAMPLE 9

### Controlled pH

Chitosan, sodium caseinate and fluorescein were mixed at pH5 by low shear mixing, followed by ultrasound. This mixture then had sunflower oil and rhodamine added and mixed by high shear mixing. This is summarised in FIG. 12.

The resulting emulsion was placed within a dialysis membrane and placed in external accepter solutions at pH3, 5 and 10. The concentration of fluorescein and rhodamine released out of the dialysis membrane was quantified over time.

FIG. 13 shows the differential release of the two compounds with pH.

### EXAMPLE 10

### Particle Fabrication

Lipid-crystal-particles were constructed by firstly forming an o/w (‘hot’) nanoemulsion at temperatures above the melting point of a crystallising lipid contained within its droplets. Temperature was then reduced to initiate crystallisation of the droplets which acted as templates for the formation of the lipid-crystal-particles. Polysaccharide/protein complexes were formed by mixing the biopolymers and then adjusting the solution's pH conditions to promote complexation. Both types of functional “Pickering” particles were developed through industrially-available processing (e.g. high shear mixers). In this project we will build on present expertise and utilise alternative species (e.g. Beta-Lactoglobulin/low-methoxyl-pectin, wax) for the fabrication of new particulate structures.

### EXAMPLE 11

### Active Encapsulation

Active incorporation within the particulate structures was previously addressed by carrying out the encapsulation step during particle fabrication. For the lipid crystals particles, the active was introduced within the initially formed nanoemulsion (prior to crystallisation) and for the polysaccharide/protein complexes, the active was added to the biopolymer aqueous mixture (prior to pH adjustment and complexation).

### EXAMPLE 12

### Triggered Active-Release

Release of the encapsulated active was triggered by inducing the collapse of the particles' structure. Increasing the temperature of the lipid crystal particles (above their melting point) or increasing the pH of the polysaccharide/protein complexes (above their isoelectric points) both resulted in immediate active release (FIG. 14).

### EXAMPLE 13

### Pickering Functionality Enhancement

The particulate structures' size and surface-activity/wettability were controlled to promote their Pickering functionality. Lipid crystals' particle size and wettability were adjusted by controlling the size of their droplet-precursors and by using small concentrations of emulsifiers. The polysaccharide/protein ratio of the biopolymer complexes was adjusted to control their size as well as their surface activity. Both particle types successfully stabilised o/w emulsions and long-term stability was maintained even for the active-containing particles (FIG. 15). This project will build on these successful approaches to enhance Pickering functionality.

### EXAMPLE 14

### Sodium Caseinate-Chitosan Complex Formation

A 25 g polysaccharide solution (1 wt % chitosan, 2 wt % acetic acid and 97 wt % water adjusted to pH 5) was added dropwise on a magnetic stirrer to a 25 g protein solution (comprising 1 wt % sodium caseinate in a 30 mM sodium acetate buffer adjusted to pH 5). These yielded complexes of sodium caseinate and chitosan (see FIG. 16). The size of the complexes could optionally be subjected to further processing (e.g ultrasound, high shear mixing, high pressure homogenisation) to yield suspensions of complexes with different z-average diameters. FIG. 1 shows a micrograph of complexes of sodium caseinate and chitosan formed under low shear:

### EXAMPLE 15

### Model Active Loading in the Complexes

A model active, fluorescein, was be loaded into the complex structure by dissolving it in the protein or chitosan solution prior to complex assembly. When the total concentration of fluorescein in the final suspension was 0.02 mg fluorescein/g complex suspension, the encapsulation efficiency (EE) depended on the total biopolymer concentration and the ratio of chitosan-to-sodium caseinate present. EE of fluorescein ranged between 60 and 90%. The encapsulation procedure could also be extended to different actives (for example, the model active rhodamine B could be encapsulated). Optionally the active could also be loaded after complex assembly with negligible change in EE.

### EXAMPLE 16

### Release of Actives Using a pH-Trigger

Release of fluorescein and rhodamine B from sodium caseinate-chitosan complexes was monitored as a function of pH with a glass pH probe. In a release experiment, 55.5 g of suspension with encapsulated active at pH 5 was stirred in a 50 mL beaker on a magnetic plate. 10% NaOH aliquots were added in pH intervals from pH 5 to pH 11. When each pH interval had been reached, a 1.5 mL aliquot was withdrawn from the suspension and transferred to an Eppendorf tube. These withdrawn samples were centrifuged at 15,000 for 60 min and active concentration in the supernatant was determined by UV-VIS. This is shown in FIG. 17.

The pH at which release occurs can be controlled by judicious selection of polysaccharide and protein. For example, whey protein isolate and sugar beet pectin have been employed to alter the active trigger pH range.

1. An emulsion comprising
(a) a continuous phase;
(b) a dispersed phase comprising an active compound (1); and
(c) colloidal particles located at the interface between the continuous phase and the dispersed phase.

2. The emulsion of claim 1 further comprising a polymer where the particles are either partially or fully surrounded by the polymer which is also located at the interface between the continuous phase and the dispersed phase.

3. The emulsion of claim 1 wherein the colloidal particles are solid lipid particles and are present at the emulsion interface or elsewhere in the system as discrete particles.

4. The emulsion of claim 1 further comprising one or more polysaccharides, one or more proteins, or a mixture of one or more polysaccharides and one or more proteins where the particles are partially or fully surrounded with the one or more polysaccharides, the one or more proteins or the mixture of one or more polysaccharides and one or more proteins.

5. The emulsion of claim 1 wherein the colloidal particles are present at the emulsion interface or elsewhere in the system as fully or partially fused particles.

6. The emulsion of claim 1 wherein, the colloidal particles are present as fully or partially associated particles

7. The emulsion of claim 1 further comprising one or more additional segregated active ingredients and independent release mechanisms.

8. The emulsion of claim 1 wherein the emulsion is an oil-in-water emulsion.

9. The emulsion of claim 1 wherein the emulsion is a water-in-oil emulsion.

10. The emulsion of claim 1 further comprising an active compound (2), where the active compound (2), is contained within the colloidal particles.

11. The emulsion of claim 1 wherein the continuous phase comprises an active compound (3).

12.-13. (canceled)

14. The emulsion of claim 1 further comprising active compounds (2) and (3) where the active compounds (1), (2) and (3) are independently selected from the group consisting of agrochemicals, pharmaceuticals, food additives, cleaning agents, personal care substances and compounds that are capable of or adapted to impart functionality upon delivery to a site of use of the emulsion.

15. The emulsion of claim 14 wherein the active compounds (1), (2) or (3), or a combination thereof, are independently selected from the group consisting of agrochemicals and food additives.

16. A method for killing, preventing or controlling the growth of a pest, the method comprising applying the emulsion of claim 15.

17. A method for controlling delivery of one or more active compounds (1), (2), or (3), or a combination thereof, to the human skin, mouth, gastrointestinal tract or another part of the human body, the method comprising combining the emulsion of claim 14 with the one or more active compounds (1), (2), or (3), or the combination thereof.

18. A method for maintaining chemical, photochemical, or physical stability of one or more of actives (1), (2), or (3), or a combination thereof, during emulsion storage, the method comprising combining the emulsion of claim 14 with the one or more actives (1), (2), or (3), or the combination thereof.

19. A method for independently controlling release of one or more of actives (1), (2), or (3), or a combination thereof, through one or more change in environmental conditions, the method comprising combining the emulsion of claim 14 with the one or more actives (1), (2), or (3), or the combination thereof.

20. The method of claim 19 wherein the change in environmental condition is selected from the group consisting of temperature, pH, radiation, ionic conditions, and osmotic stress, and a combination thereof.

21. A process for preparing the emulsion of claim 8 comprising the step of emulsifying an oil into an aqueous lipid suspension so as to provide an emulsion of oil droplets in water stabilized by discrete lipid particles at the oil-water interface.

22. A process for preparing the emulsion of claim 9 comprising the step of emulsifying an aqueous lipid suspension into an oil so as to provide an emulsion of aqueous droplets in oil stabilized by discrete lipid particles at the water-oil interface.
